(19) United States
(12) Patent Application Publication
(10) Pub. No.: US 2005/0118595 A1
(43) Pub. Date: Jun. 2, 2005

(54) Fabrication of surfaces with reduced protein adsorption and/or cell adhesion

(76) Inventor: Joerg Lahann, Ann Arbor, MI (US)

Correspondence Address: Joerg Lahann, 2155 Georgetown Boulevard, Ann Arbor, MI, 48105, US

(21) Appl. No.: 10/499,768

(22) PCT Filed: Dec. 19, 2002
(86) PCT No.: PCT/US02/40751

(30) Foreign Application Priority Data
Dec. 19, 2001 (DE) 101 62 435.2

(51) Int. Cl. A61L 2/00; C12Q 1/68; G01N 33/53

(52) U.S. Cl. 435/6; 435/7.1; 427/2.11

(57) ABSTRACT

Fabrication of surfaces with reduced protein adsorption and/or cell adhesion comprising a vapor deposition coating process such that the coating includes polymer interfaces containing chemical groups reducing the protein adsorption and/or cell adhesion. The invention allows precise anchoring and presentation of biomolecules in their biological context. The resulting systems comprise superior surfaces for design of protein- or cellbased bioassays, because of high-signal-to-noise ratios. Background adsorption is suppressed while specific interaction with capturing molecules is not affected.

The invention relates to biologically resistant surfaces, i.e. surfaces with reduced protein adsorption and/or cell adhesion providing at least parts of a substrate with a functionalized polymer. The entire functionalized polymer or parts thereof either contain functional groups that per se reduce the protein adsorption and/or cell adhesion, or contain functional groups that support subsequent chemical conversion with molecules that reduce the protein adsorption and/or cell adhesion. The functionalized polymer is prepared by forming monomers in the gas phase from xylylene or quinodimethane derivatives at elevated temperature and reduced pressure, then polymerizing at reduced temperature by cooling. The monomer-forming temperature and pressure are 500-1000° C. and <500 Pa, depending on the starting material. The invention further relates to the field of polymer coating made by chemical vapor deposition (CVD) and the use of homogenously distributed functional groups for defined surface design.

The invention further relates to applications of said biologically resistant surfaces for bioassays for screening of proteins or cells. Proteins in the sense of the invention include all biomolecules. Specific classes of biomolecules include, but are not limited to Biomolecules in the sense of the invention include, but are not limited to peptides, amino acids, proteins, DNA, RNA, nucleotides, adenosine, thymidine, guanosine, cytidine, uridine, deoxyadenosine, deoxythymidine, deoxyguanosine, and deoxycytidine, nucleoside analogs (e.g., 2-aminoadenosine, 2-thiothymidine, inosine, pyrrolo-pyrirmidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages), growth factors, cell adhesion molecules including tenascins, astrotactin, cintactin, NrCAM, neurofascins, L1, Neuroglian, TAG-1, anxonin-1, fascilins, cadherins, selectins, ephirins, netrins, semaphorines, chemokines, interleucines, neurotrophines, neurotransmitters, catecholamin receptors, growth factor receptors, amino acid receptors or derivatives thereof, cytokine receptors, extracellular matrix molecule receptors, integrins, integrin receptors, cell response modifiers such as chemotactic factors, hormones, hormone receptors, antibodies, lectines, cytokines, leptines, serpines, enzymes, proteases, kinases, sulfotransferases, metalloproteases, phosphatases, hydrolases, transcription factors, DNA binding proteins or peptides, RNA binding proteins or peptides, cell surface antigens, vaccines, haptens, toxins, interferons, ribozymes, anti-sense agents, plasmids, virologe proteins each as HIV-protease or hepatitis C virus protease, biological ligands, receptors, polysaccharides, lipids, antibodies, haptenes, nucleoproteins, glycoproteins, lipoproteins, steroids, or phages whether naturally-occurring or artificially created (e.g., by synthetic or recombinant methods).

Cells in the sense of the invention are all eucharyotic or prokaryotic virologe cells including human, murine, rodent, or bovine cells, cell accumulates, tissue, tissue fragments, cell colonies, bacteria, cell lysates, or cell parts such as organelles, cell subunits, and cell membranes. Cell features in the sense of the invention are biomolecules expressed by a cell that are characteristic for the expressing system. Cell features can be expressed at the surface of the cell or may be released by the cell into a surrounding medium.

Surfaces with reduced protein adsorption and/or cell adhesion have potential use for screening of a quantity of biologically active molecules, such as small molecules, DNA, proteins, or sugars and are in immediate context with pharmaceutical technologies in the fields of drug discovery, proteomics, genomics, high-throughput screening, and clinical diagnostics. They may also be suitable for design of DNA-, protein- or cell-based assays of various formats. Classes of drugs that can be used in the practice of the present invention include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents.

A more complete listing of classes and specific drugs suitable for use in the present invention may be found in “Pharmaceutical Substances: Syntheses, Patents, Applications” by Axel Kleemann and Jurgen Engel, Thieme Medical Publishing, 1999 and the “Merck Index: An Encyclopedia of Chemicals, Drugs, and Biologicals”, Edited by Susan Budavari et al., CRC Press, 1996, both of which are incorporated herein by reference.

The number of clinical compounds that are available for clinical studies has dramatically increased over the last years due to successes in combinatorial chemistry. In parallel, a vast number of potential target molecules are identified due to recent developments in genomics. Both, the availability of large drug libraries and the increased number of new target molecules have created a demand for novel technologies for screening of biologically relevant molecules. For parallel screening of a quantity of biomolecules, a large number of target molecules is generally confined to a substrate and a solution containing biomolecules is conducted with the surface. Current techniques often rely on microwell plates with sample volume in the microliter range. This requires high sample volumes, high costs and time-consuming processes. Miniaturized chips are developed for screening of biological material (e.g. U.S. Pat. No. 5,412,087, U.S. Pat. No. 5,445,934 and U.S. Pat. No. 5,744,305) and find wide application. Microdevices for in vitro screening potentially require smaller sample volume and may provide access to more efficient studies of biomolecules. The field of microfluidics has witnessed astonishing advances over the last few years such as the development of micro total analysis systems (μTAS), microfabricated cell sorters, or microseparators for DNA and proteins. Recently, microfluidic devices have been used for continuous-flow cell-based assays. However, current microdevices lack the availability of surfaces that are compatible with screening methods in proteomics or cell-assays. This is even more critical as microdevices are characterized by high surface-to-volume ratios when compared to conventional systems.

A critical feature of microassays is the control over ligand presentation and density at surfaces within a biologically inert background. SAMs of polyethylene glycol-containing thiolates are described that had reduced protein adsorption and bacteria or cell adhesion when deposited onto gold surfaces. Recently, a method is disclosed that used confinment of polymers to SAMs adsorbed on gold to fabricate films with with reduced protein adsorption and/or cell adhesion (Whitesides et al., Langmuir 2001). The preparation of the films included (i) depostion of a gold film to a substrate, (ii) self-assembly of mercaptohexadecanoic acid onto gold, (iii) chemical converrsion of the acid groups into anhydride groups, (iv) reaction of the anhydride groups with primary amino groups of polyamines, (v) acetylation of the remaining amino groups. Other approaches include the reaction of polyamines confined to SAMs with PEGs or polysaccharides.

All this methods have important features in common, including the following: (i) they require complex multi-step preparation, (ii) they are restricted to a very small group of substrates usually gold, (iii) the multi-step reactions often involve harsh chemicals and solvents that are not compatible with biological systems, and therefore (iv) the precious accession of biological processes is intrinsically restricted. This creates a great need for simple, routine methods for the fabrication of surfaces with reduced protein adsorption and/or cell adhesion on a wide range of substrate materials.

EP 665340B1 reports surface modification of a polymer device by incubation with harsh chemicals. The resulting functional groups are then used for further modification. Other methods for surface modification of materials are plasma aching and plasma polymerization (see Yasuda or EP 0519087 A1), laser treatment, or ion beam treatment. The underlying mechanisms are often poorly understood and these methods are characterized by side reactions including the fabrication or incorporation of potentially harmful chemicals.

Rather than pure surface modification, surface coating is the method of choice for some applications. Surface coating methods include carbon like diamond coatings (CLD), carbon nitride coating, deposition of several metal layers or simple spin, dip, or spray coating of polymers. CVD polymerization coatings of paracyclophane or chlorine derivatives thereof, applied in order to achieve inert surfaces (Swarc, Gorham, Union Carbide) have excellent homogeneity, adhesion and stability. Recently CVD coating of functionalized paracyclophanes has been used in order to immobilize bioactive proteins (Lahann Biomaterials 2001, Höker, D E 19604173 A1). This coating procedure developed to be a one-step coating and functionalization method offers a wide range of applications since good bulk properties of a material has been maintained combined with enhanced contact properties. The ‘activation’ of surfaces with bivalent spacer molecules offers the opportunity of further modification such as drug immobilization. By using the interfaces for immobilization of proteins, cell receptors, cytokines, inhibitors etc., bioactive surfaces that interact with the biological environment in a defined and active matter can be achieved.

PCT/US99/15968 discloses arrays of protein-capture agents, which are useful for the simultaneous detection of a plurality of proteins. The arrays comprise a thin organic layer that is between 10 and 20 nm thick. The use of monomolecular dimensioned interlayer is associated with disadvantages described for self-assembled monolayers. SAM's are restricted to a few substrate materials. Porous structures such as foams, scaffolds or membranes are difficult to process and applications in chemically aggressive environments such as in vivo are not possible. The herein disclosed methods allow for overcoming these drawbacks.

U.S. Pat. No. 6,103,479 (Taylor) discloses miniaturized cell array methods and apparatus for cell-based screening. These devices can be used with methods of performing high-throughput screening of the physiological response of cells to biologically active compounds and methods of combining high-throughput with high-content spatial information at the cellular and sub-cellular level as well as physiological, biochemical and molecular activities.

Other prior art references, which generally describe cell arrays and methods and apparatus to use the same include WO 01/07891 (Kapur et al.), WO 00/60356 (Kapur et al.), U.S. Pat No. 5,776,748 (Singhvi et al.), and WO 00/53625 (Rossi et al.). However, all of these references include multi-step processes and include the use of solvent.

U.S. Pat. No. 6,192,168 (Feldstein et al.) describes a reflectively coated optical waveguide and fluidics cell integration, which includes a waveguide having a patterned, reflective polymer.

Arrays of protein-captive agents are useful for simultaneous detection of a plurality of proteins which are expression products or fragments thereof, of a cell or population of cells as described in PCT WO 00/04389. The arrays are useful for various proteomic applications including assessing patterns of protein expression and modification in cells.

### SUMMARY OF THE INVENTION

The fabrication of surfaces with reduced protein adsorption and/or cell adhesion comprises coating at least parts of the surface with a functionalized polymer. The entire functionalized polymer or parts thereof either contain functional groups that per se reduce the protein adsorption and/or cell adhesion, or contain functional groups that allow subsequent modification with molecules that reduce the protein adsorption and/or cell adhesion. The functionalized polymer is prepared by forming monomers in the gas phase from xylylene or quinodimethane derivatives at elevated temperature and reduced pressure, then polymerizing at reduced temperature by cooling. The monomer-forming temperature and pressure are 500-1000° C. and <500 Pa, depending on the starting materials. The surfaces with reduced protein adsorption and/or cell adhesion are useful for several applications such as the manufacturing of protein or cell arrays, immobilization of drugs for tissue engineering, micro-reactors, surfaces for protein or DNA screening or electro-optical devices.

When chemically addressable surfaces are needed for the fabrication of microdevices for parallel analysis of biomolecules, CVD polymerization of functionalized [2.2]paracyclophanes can be utilized. The technique has been used for coating of several materials with polymers. Since the coating step is substrate-independent, the technology provides a generic approach to microstructuring of microdevices. While overcoming restrictions associated with gold/alkanethiolates-based techniques, the technology maintains intrinsic advantages of soft lithography, e.g. accuracy, broad availability, and low costs.

It is an object of this invention to provide a one step CVD process resulting in functionalized polymers that are biologically resistant.

It is another object of this invention to provide the functionalized polymer on essentially any shaped three dimensional or porous structures.

It is another object to provide a simple, inexpensive quick scale-up method of producing a biologically resistant and chemically addressable surface. Additionally it is another object to provide applications for the herein disclosed methods.

### DESCRIPTION OF THE PREFERRED EMBODIMENT

Universal applicability of the CVD polymer to various substrates, such as polymers, metals or composites makes the procedure described below attractive for fabrication of microdevices for parallel analysis of biomolecules. Generally, the polymer films contain functional groups that are capable to reduce adsorption of proteins and/or adhesion of cells. The monomer units may be achieved either by thermal or photochemical activation of suitable precursors (usually paracyclophanes) in a CVD process. All interfaces are based on poly(para-xylylene) polymers, polymer derivatives or copolymers thereof. The interface is built up by polymers that contain one or more different repetition units, where at least one of the repetition units is selected from the class of repetition units with the general structure 1 (as shown below), while other repetition units can be variably designed, although para-xylylene is the mainly suitable other repetition unit. 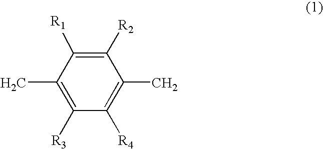
Rₙ (n−1,2,3,4) may be equal or different and may be selected from the group consisting of hydrogen, C1-C4 alkyl, aryl, amine, alcohol, ether, ethylene glycol, cyclic ether, thioether, crown ether, primary amide, secondary amide, ethylene glycol containing primary amide, ethylene glycol containing secondary amide, urethane, nitrile, isonitrile, nitrosamine, lactone, ethylene glycol containing urethane, carbamate, ethylene glycol containing carbamate, lactam, imine, hydrazone, ester, ethylene glycole containing ester, nitro compounds, nitrile, halo, organic radical, metalized group, acid halide group, isocyantate, thioisocyante, sulfur-containing groups (e.g. sulfonic acid, thioether, sulfonate, or sulfate ester group), silicon-containing group (e.g. silyl or silyloxy), or sugar derivatives.

Depending on the used polymer, the required temperatures for monomer creation are between 400 and 1000° C. and the pressures are below 500 Pa.

The proposed procedure for coating of devices with functionalized polymers provides an increased surface concentration of functional groups with a defined and controlled ratio when compared to conventional methods such as plasma treatments. Due to the rigid background of the deposited polymer aging effects as a consequence of interactions with analyte solutions can be neglected.

In a preferred embodiment, the surfaces contain functional groups that per se reduce the protein adsorption and/or cell adhesion. Such functional groups may include but not be restricted to methoxy, ethoxy, hydroxyethoxy, hydroxymethoxy, ethylene glycol, oligoethylene glycol, amide, ester, sugar, mannitol, sorbitol, peralkylated mannitol, peralkylated sorbitol, amino acids, peptides, cyclic ethers, ethers, amines, urethanes, or carbamates. The polymer coating that reduces protein adsorption and/or cell adhesion will include repetition units that include above-mentioned functional groups. Structures 2 to 66 are examples of suitable repetition of the disclosed polymers, although other suitable repetition units are possible, as long as they reduce protein adsorption and/or cell adhesion by at least 50% as compared to a monolayer coverage of the protein, wherein
Me is methyl or hydrogen,
m is selected from 0 or 1;
n is selected from 0, 1, 2, or 3;
X is selected from the group consisting of amides, amide derivatives, cyclic ethers, sugar derivatives, amines, amine derivatives, amino acids, amino acid derivatives, carbamates, carbonates, and nitrites;
R₁, R₂, R₃, R₄ may be equal or different and are selected from the group consisting of hydrogen, C1-C4 alkyl, aryl, ether, ethylene glycol, cyclic ether, thioether, alcohol, crown ether, lactam, lactone, amine, imine, hydrazone, ester, nitro, nitrile, halogene, acid halide group, isocyantate, thioisocyante, thioether, ketone, or sulfonate; and
R₅, R₆, R₇, R₈ may be different or equal and are selected from the group consisting of hydrogen, phenyl, and C₁-C4 alkyl.

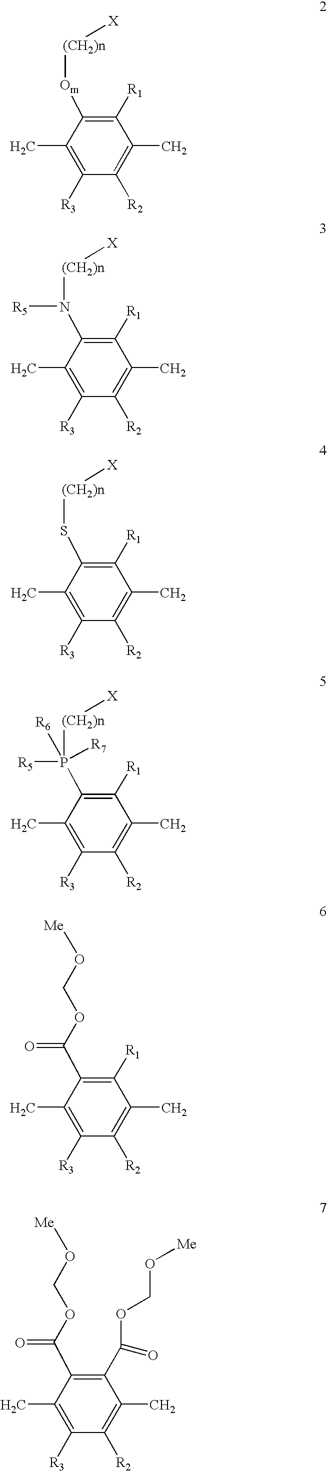

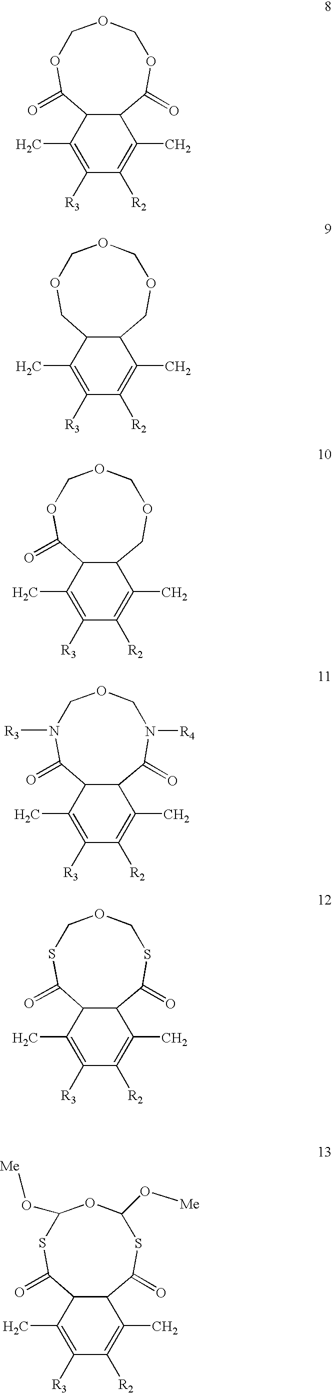

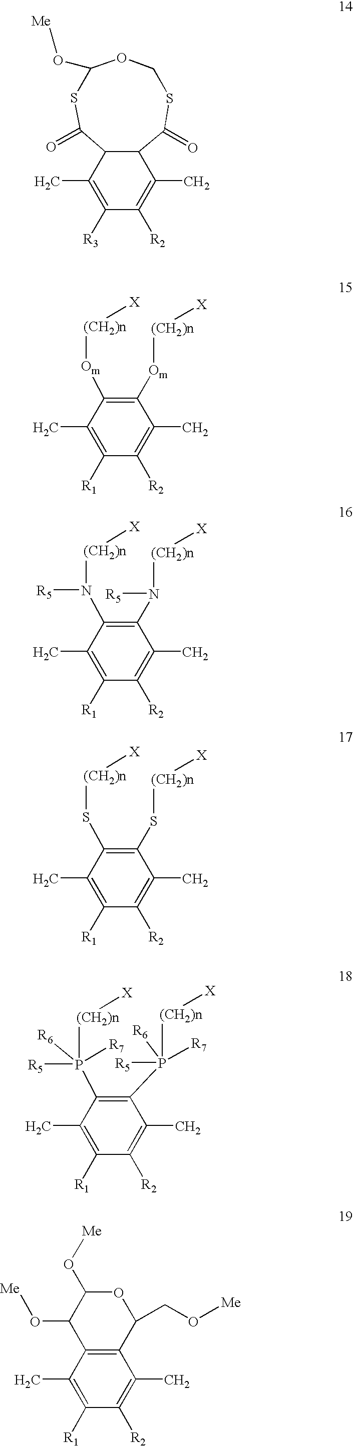

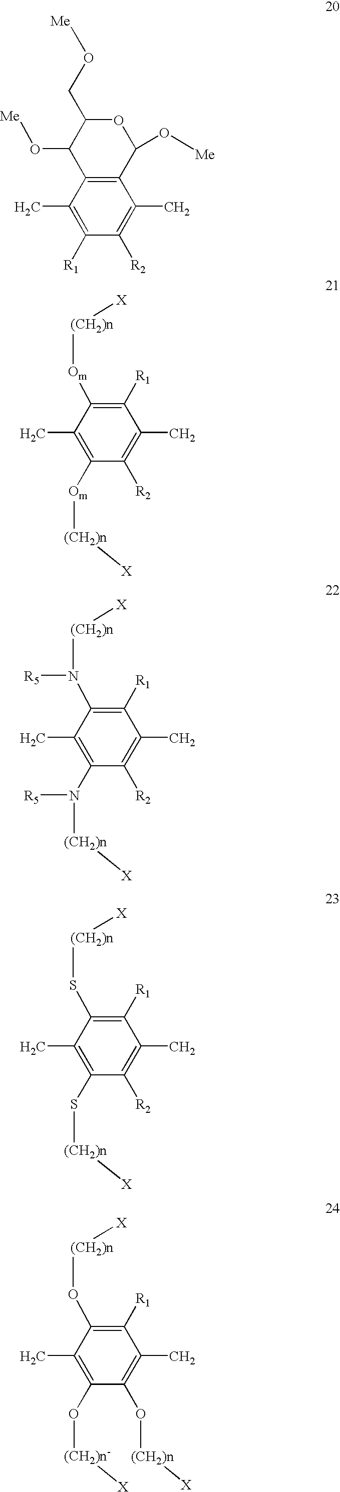

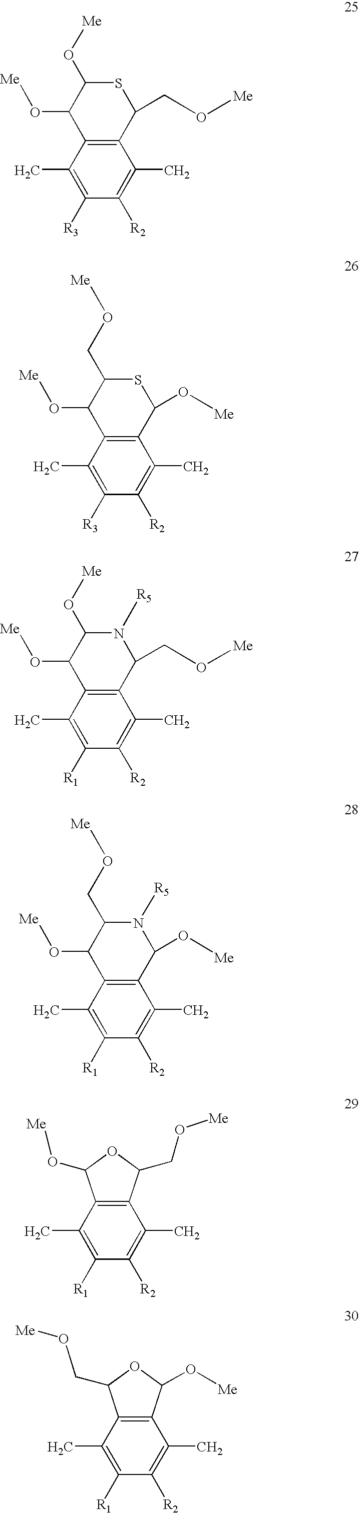

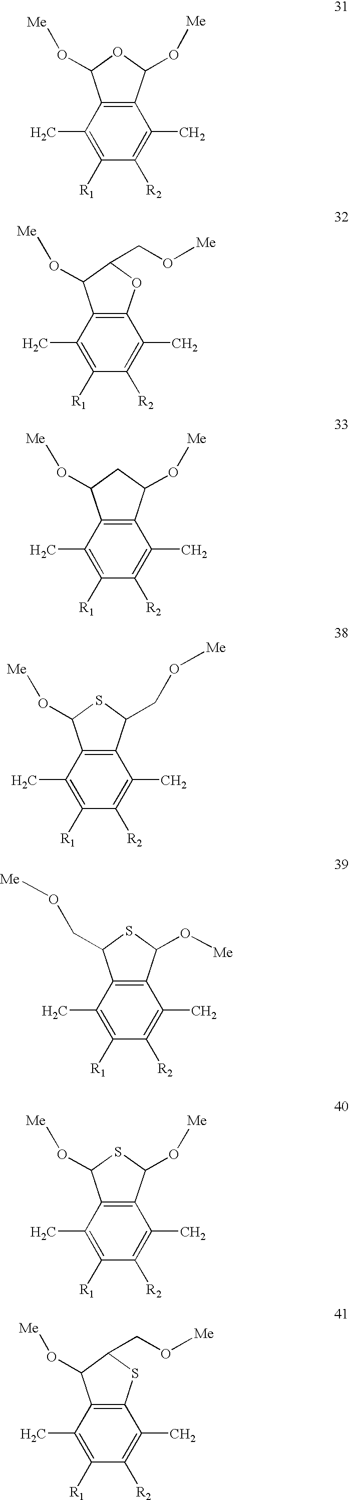

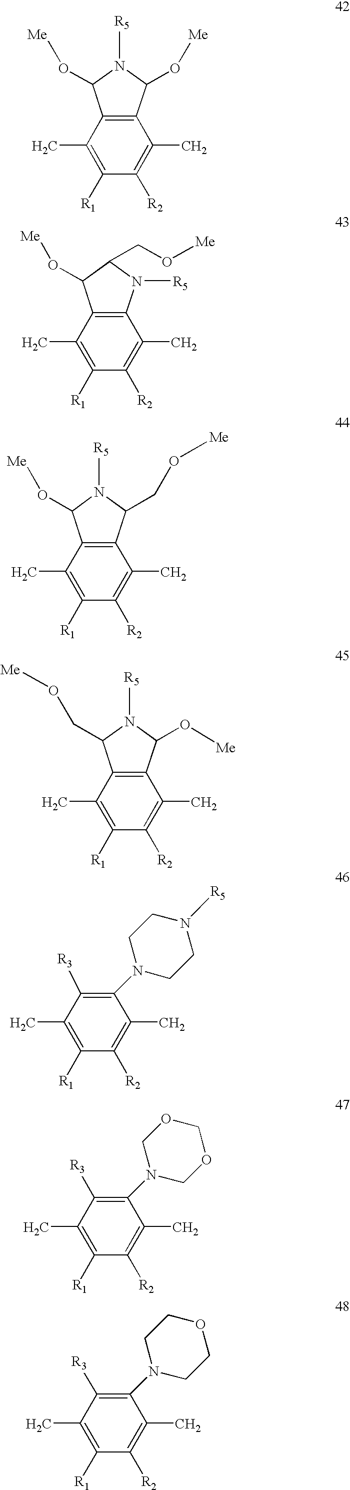

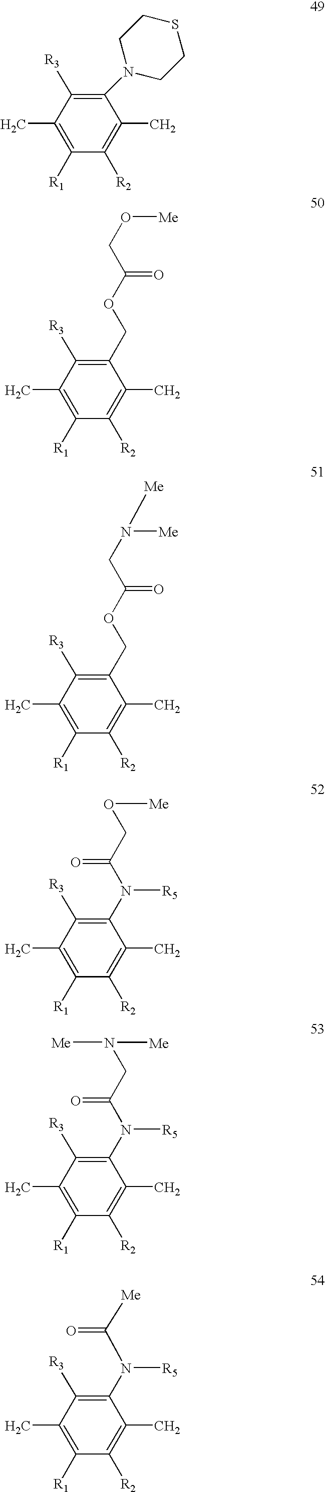

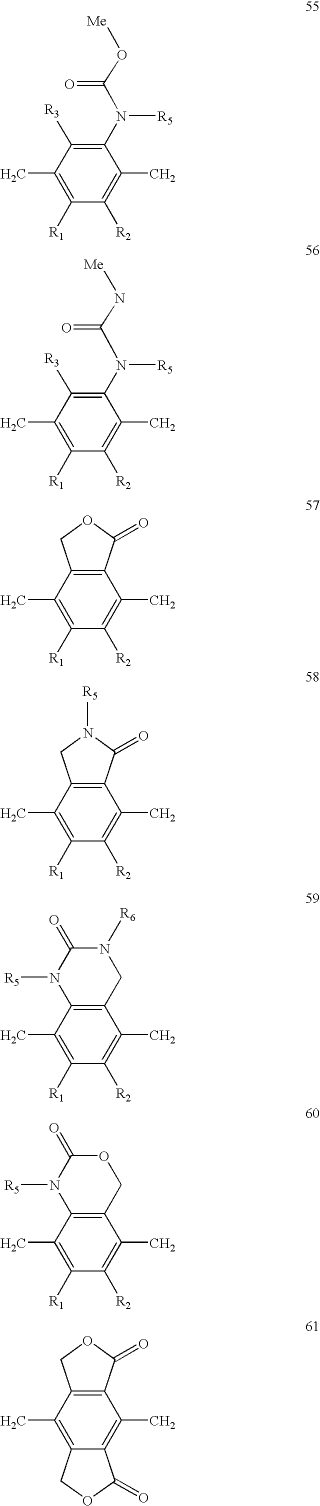

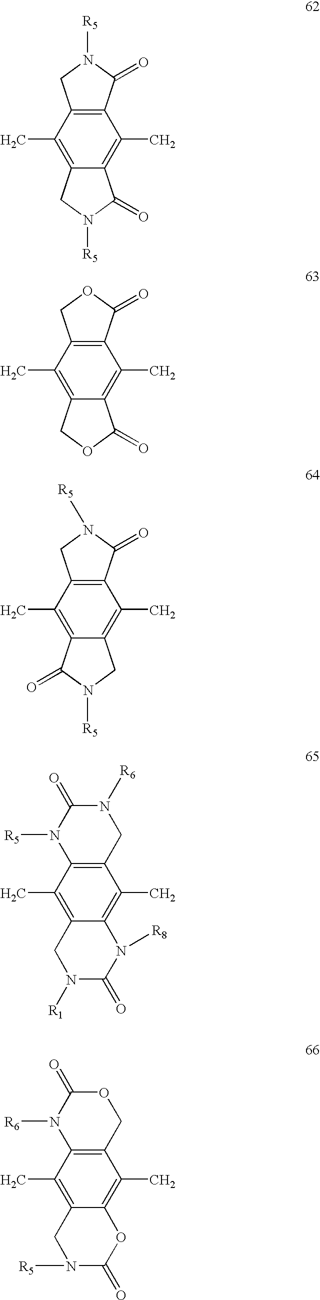

In another preferred embodiment, the surface contains functional groups that have sufficient intrinsic reactivity to react with molecules that reduce protein adsorption and/or cell adhesion resulting in stable chemical linkages. In this case, the polymer coating must not per se reduce protein adsorption and/or cell adhesion, as long as its functional groups support conversion with molecules that enable reduction of protein adsorption and/or cell adhesion, such as polyethylene glycols, oligoethylene glycols, or ethylene glycols (all unbrunched, brunched, dentrimeric, or cyclic); polyamines, oligoamines, or amines (all unbrunched, brunched, dentrimeric, or cyclic); polysaccharides, oligosaccharides, or sugars (unbrunched, brunched, dentrimeric, or cyclic), mannitol, mannitol derivatives, such as peralkylated mannitol, sorbitol, sorbitol derivatives, such as peralkylated sorbitol, amino acids, proteins, peptides, DNA, metals, transition metals, surfactants, tensides, phospholipids, lipids, steroids, antibiotica, interleucines, prostaglandines, urethanes, oligourethanes, or polyurethanes (unbrunched, brunched, dentrimeric, or cyclic), polyacrylamides, polymethacrylamides, crown ethers, polyethers, oligoethers or ethers (unbrunched, brunched or dentrimeric), polycarbonates, oligocarbonates, or carbonates, carbamates, ketones, or aldehydes. A vast number of molecules that reduce protein adsorption and/or cell adhesion are known to a person skilled in the art and a selection might be found in WO 02/06407.

The reaction of the functional groups of the functionalized polymer with the drug may optionally take advantage of bivalent linker molecules. The reaction of the interface with the drug may also be carried out in aqueous solution ideal for applications associated with cells or biomolecules. The resulting polymer surfaces—whether per se reducing adsorption of proteins and/or cell adhesion or after subsequent modification—present an biologically inert background that can be used for fabrication of biological relevant devices, such as protein- or cell-assays.

According to the invention, a surface effectively reduces protein adsorption and/or cell adhesion when the surface shows at least 50% lower adsorption (or adhesion) than the monolayer coverage of the studied protein (or cell). Protein adsorption may be assessed by surface plasmon spectroscopy, while cell adhesion may be measured by fluorescence staining and fluorescence microscopy. In a more preferred embodiment, the reduction will be at least 90%; in an even more preferred embodiment, the reduction will be at least 95% and in some cases reduction of protein adsorption and/or cell adhesion might be higher than 99%.

In a further embodiment, remaining or additionally created functional groups can further be used for immobilization of capturing biomolecules. Capturing biomolecules shall comprise those molecules that are confined to the surface and bind at least a part of the cells, cell parts, organelles, or biomolecules that are subject to screening. Capturing biomolecules include, but are not limited to biological ligands, receptors, antibodies, haptenes, lectines, carbohydrates, DNA, RNA, artificial receptors. A variety of capturing biomolecules is known to an expert to the field some of them disclosed in WO 00/04390. The binding event between capturing biomolecules and biomolecules allows the isolation of a given sort of biomolecules. Therefore, the capturing biomolecules must posses selectivity towards a specific biomolecule or at least a class of biomolecules. Suitable binding pairs can be antibody/antigene, antibody/heptene, enzyme/substrate, integrin/cell receptor, biomolecule/cell, cell/cell, carrier protein/substrate, lectine/carbohydrate, receptor/hormone, receptor/cytokine, protein/DNA, protein/RNA, peptide/DNA, peptide/RNA, two DNA single strains, DNA/RNA, DNA/DNA, where either of both partners of these couples may serve as capturing biomolecule.

The describe microdevices are especially useful for screening or high-throughput screening of biomolecules among a biological class of biomolecules. Those biological classes include growth factors, neurotransmitters, catecholamin receptors, growth factor receptors, amino acid receptors or derivatives thereof, cytokine receptors, extracellular matrix molecule receptors, integrins, integrin receptors, hormones, hormone receptors, antibodies, lectines, cytokines, leptines, serpines, enzymes, proteases, kinases, phosphatases, hydrolases, transcription factors, DNA binding proteins or peptides, RNA binding proteins or peptides, cell surface antigenes, virologe proteins such as HIV-protease or hepatitis C virus protease, or phages.

For this purpose, polymer films consisting of co-polymers with more than one repetition unit or polymers that contain repetition units with more than one type of functional groups may be useful. For instant, a typical co-polymer may include two different functional groups, where one group is provided in excess over the other. Typical ratios may include, but not be limited to the group comprising 10:1, 100:1, or 1000:1.

While the functional group representing the majority will be used to reduce unspecific protein adsorption and/or cell adhesion (i.e. establishment of the biologically resistant background), the minority group is used to introduce specific capturing sides into the surface. This results in platforms for bioassays with enhanced signal-to-noise ratios because of the low background adsorption, so that the observed signal is mainly generated by the captured analyte molecule.

In another preferred embodiment, the functional group that represents the majority stems from repetition units that reduce protein adsorption and/or cell adhesion, such as repletion units of the structures 2 to 66. The functional group that represents the minority may than be picked form those functional groups with high chemical reactivity to enable binding of the capturing biomolecule. Corresponding repetition units are known from US 09/912166. Examples of those repletion units are listed below as structures 67 to 82.

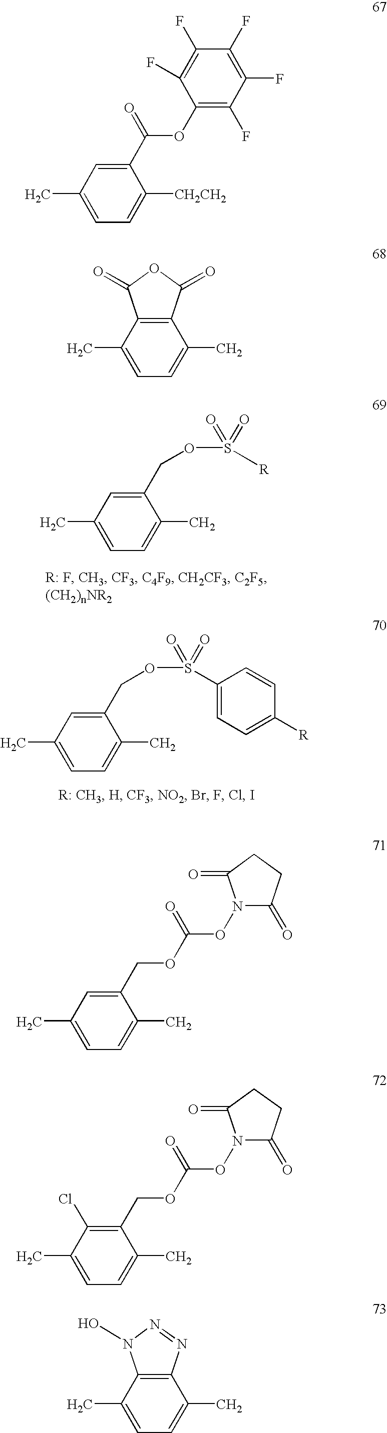

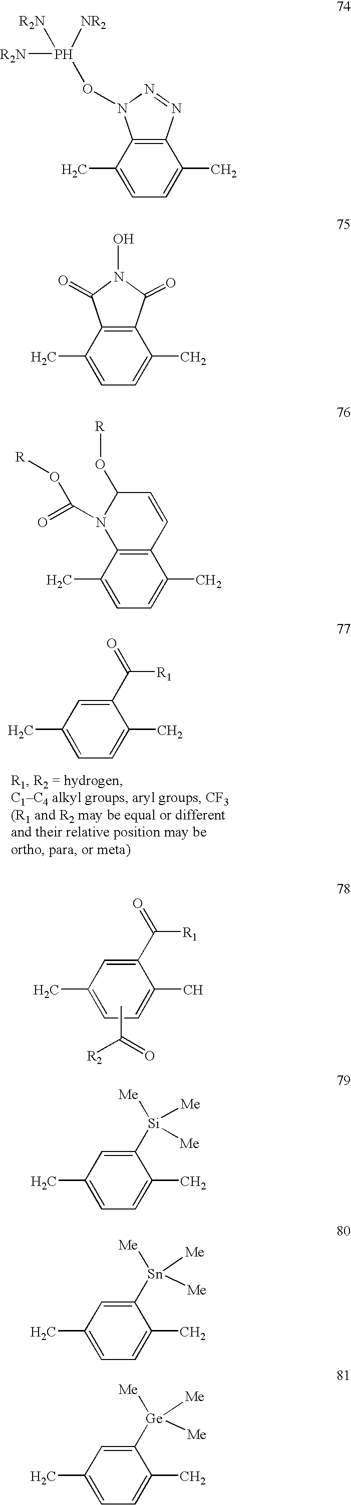

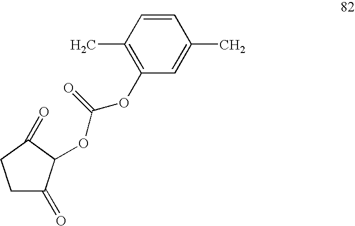

In another embodiment, the invention deals with the preparation of the functionalized polymer coatings. Functionalized polymers may be prepared from molecules of the following structure, 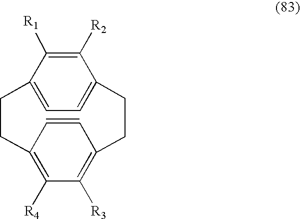
wherein Rₙ (n=1,2,3,4) may be equal or different and may be selected form the group consisting of hydrogen, C1-C4 alkyl, aryl, anhydride, amine, thiole, nitrile, amide, methoxymethyl, acetylmethyl, trifluoroacetylmethyl, trifluoroacetyl, hydroxymethyl, hydroxymethoxymethyl, bromide, iodide, chloride, lacton, pentafluorphenol ester, triflate, tosylate.

However, suitable polymers are not restricted to [2.2]paracyclophanes, but can also be prepared from functionalized p-xylylenes, such as α,α-disubstituted and α,α′-disubsituted functionalized p-xylylenes, functionalized quinodimethanes, functionalized p-quinone methids, functionalized p-quinone diimines, functionalized p-quinone methide imines, functionalized aromatic bisketenes, or functionalized p-quinones; as long as the their polymerization results in polymers with the proposed repetition units in sufficient yield, purity and without incorporation of toxic side products. General groups of precursors for CVD polymerization are known to an expert in the field and examples are given by Itho (Prog. Polym. Sci., 2001).

In another embodiment, functionalized polymers may be prepared by co-polymerization of precursors of the general structure 83 with precursors of the general structures 84 and/or 85. Those co-polymers are found to be suitable functionalized polymers for biologically resistant surfaces. They further allow specific tailoring of physical and/or chemical surface properties including topology as required by a given application. 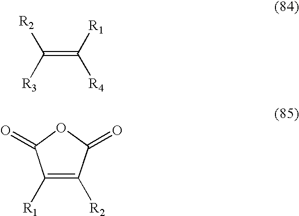
wherein Rₙ (n=1,2,3,4) may be equal or different and may be selected from the group consisting of hydrogen, C1-C4 alkyl, aryl, amine, alcohol, ether, ethylene glycol, cyclic ether, thioether, crown ether, primary amide, secondary amide, ethylene glycol containing primary amide, ethylene glycol containing secondary amide, urethane, nitrile, isonitrile, nitrosamine, lactone, ethylene glycol containing urethane, carbamate, ethylene glycol containing carbamate, lactam, imine, hydrazone, ester, ethylene glycole containing ester, nitro compounds, nitrile, halo, organic radical, metalized group, acid halide group, isocyantate, thioisocyante, groups of the general nature CO(O-M-A) (with M: C1-C4 aliphatic or aromatic group and A: e.g. hydrogen, hydroxyl-, amino-, or carboxy groups), sulfur-containing groups (e.g. sulfonic acid, thioether, sulfonate, or sulfate ester group), silicon-containing group (e.g. silyl or silyloxy), or sugar derivatives.

In another embodiment of the invention, the polymer coating may be used to bind artificial or natural molecules that change the surface properties of the microdevice or increase the surface area exposed to the analyte solution. Useful molecules include hydrophobic molecules as well as hydrophilic molecules, such as hydrogels. Especially, temperature-sensitive materials, such as poly(N-isopropylacrylamide), ethylene oxide-propylene oxide co-polymers (e.g. Pluronics©), or temperature sensitive proteins or peptides—natural or synthetic—can be bound to the surface via functionalized polymer coating.

In another embodiment of the invention, spacer systems may be used to bind molecules. Spacers include but are not restricted to diisocyantes, dicarbxylic acid chlorides, dioles, diamines, dithiols oder dicarboxylic acids and their active esters. A spacer is any molecule that allows for chemical connection between surface and target molecule. The binding occurs via chemical interactions, such as covalent bonding.

Due to the mild character of the deposition process, side reactions are suppressed and the deposited films are homogeneous with respect to their chemical structure and topology—unless otherwise intended. Gradients may be achieved by establishment of temperature gradients at the substrate being subject to deposition of the functionalized polymer. Another advantage of the disclosed method is that straightforward synthesis and selection of appropriate precursor allows the establishment of different functional groups beside each other. This feature is especially crucial, when immobilization of more than one type of biomolecules to the same substrate is intended. Spatially directed immobilization of biomolecules becomes than possible. Furthermore, the disclosed invention provides a defined chemical surface even to those devices that are composites of different starting materials.

When depositing polymers from precursors based on the general structure (1), temperatures between 400 und 900° C. and pressures below 150 Pa were suitable for activation of the precursor, while deposition is best conducted at temperatures below 160° C. The deposited polymers allow binding of biomolecules—direct or via spacers. The functionalized polymer can be used for devices made of different materials, such as polymers, composites, silicon, semiconductors, glass, or metal.

Furthermore, the once deposited film may be subject to further modification using conventional surface modification methods, such as plasma etching with gas or vapor plasma, such as oxygen, water, ammoniac, argon, sulfur dioxide, nitrogen, hydrogen plasmas or mixtures thereof.

Prior to coating in the vapor deposition process, pretreatment of the substrate may be used to improve adhesion behavior. The method of choice is mainly depending on the type of substrate and all methods known to a person skilled in the field of adhesion improvement may be applied. Especially a pretreatment with cold gas plasmas, such as oxygen, hydrogen, nitrogen, ammoniac, carbon dioxide, ethylene, acetylene, propylenes, butylenes, ethanol, acetone, sulfur dioxide or mixtures thereof have proven themselves to be advantageous in improving the adhesion behavior of the deposited polymer coatings.

According to another embodiment of the invention, surface patterns can be designed, so that only certain areas of the substrate contain groups that reduce the adsorption of proteins and/or the adhesion of cells. The patterns are achieved by (1) depostion of a polymer film consisting of poly[para-xylylene carboxylic acid pentafluorophenolester-co-N-(methoxymethyl)para-xylylamide] and (2) microcontact printing (μCP) of amino-derived biotin-ligands and (3) reaction of the remaining regions with 2-(aminoethoxy)ethanol. Since the first step involves the coating of the surface, the procedure is independent from the substrate material. The polymer film is homogenously deposited on the substrate by means of CVD co-polymerization of [2.2]paracyclophane 4-carboxylic acid pentafluorophenolester and 4-N-(methoxymethyl)amido [2.2]paracyclophane. [2.2]paracyclophane is converted to 4-trifluoroacetyl [2.2]paracyclophane by reaction with trifluoroacetic acid anhydride and aluminum chloride in dichloromethane. Subsequently, 4-trifluoroacetyl [2.2]paracyclophane is heated under reflux for 4.5 h in aqueous KOH (10% (w/v)) to yield 4-carboxy [2.2]paracyclophane. 4-carboxy [2.2]paracyclophane is then reacted with oxalylic chloride and the resulting 4-chlorocarboxy [2.2]paracyclophane is allowed to react with aminomethyl methylether to yield 4-N-(methoxymethyl)amido [2.2]paracyclophane. To seal a substrate with the reactive polymer, a 1:10-mixture (w:w) of [2.2]paracyclophane 4-carboxylic acid pentafluorophenolester and 4-N-(methoxymethyl)amido [2.2]paracyclophane is pyrolyzed in the vapor phase to form the corresponding para-quinodimethanes, which condensed onto the substrate and spontaneously polymerized. During the polymerization, the temperature of the substrate is controlled to be below 45° C. The relatively low substrate temperature allows coating of temperature-sensitive substrates (e.g. poly(lactic acid)) without decomposition. The pyrolysis temperature was found to be crucial for the quality of the reactive polymer: pyrolysis is best conducted at a temperature of 570° C. When synthesized under these conditions, the chemical composition of the resulting polymer film is in good accordance with the theoretically expected values as determined by X-ray photoelectron spectroscopy (XPS). μCP is used to pattern substrates with a set of parallel lines of (+)-biotinyl-3,6,9-trioxaundecanediamine with a width of 50 μm. The lines are separated by 50 μm wide regions of 2-(aminoethoxy)ethanol. A biotin-based ligand is chosen, since its strong non-covalent interaction with streptavidin (K_{D}=10⁻¹⁵ M) allows the patterning of streptavidin on the surface. μCP is done by means of a PDMS stamp that is previously oxidized in an air plasma. Within 5 min after plasma treatment, the PDMS stamp is inked with an ethanol solution of (+)-biotinyl-3,6,9-trioxaundecanediamine and the stamp is gently pressed onto the coated substrate. Remaining pentafluorophenol groups are now allowed to react with 2-(aminoethoxy)ethanol to create surface regions that reduce protein adsorption and/or cell adhesion. This treatment effectively inhibited non-specific adsorption of proteins and endothelial cells. Generation of cell patterns is achieved subsequently by layer-by-layer self-assembly on patterned substrates. Streptavidin is used as a linker as it bound selectively to the biotin-exposing areas. The use of streptavidin as a linker generates a universal platform for further attachment of biotin-conjugated proteins, since streptavidin has two pairs of binding sites on opposite faces. Two of its binding sites at one face are used to link the protein to the biotin-coated regions of the surface, leaving two binding sites on the opposite face for further attachment. Biotin-conjugated human anti-as-integrin is then used to create a cell-binding surface. Its immobilization is achieved by incubation of the streptavidin-pattemed sample with a solution of human anti-α₅-integrin. The antibody exclusively bound to the biotin/streptavidin-modified surface areas. Its high selectivity is proven using a fluorescein-labeled secondary antibody that recognizes the heavy chain of human anti-α₅-integrin. Cell studies confirmed the spatially controlled layer-by-layer self assembly on the reactive polymer. By patterning substrates into regions that alternately promote or prevent the binding of human anti-α5-integrin, the attachment and spreading of BAECs is controlled.

Potential Applications may include coating of intra- and/or extracorporeally useable biomedical devices, such as catheters or implants, materials for tissue engineering or neuroelectronics, cell assays, such as static or dynamic versions of cell motility, cell differentiation, cell proliferation, cell vitality, cell adhesion, cell metabolism, cell migration, or cell invasion assays. Further applications include enzyme activity assays, enzyme-linked immunoassays, epitop mapping assays or capturing assays for biomolecules, such as growth factors, neurotransmitters, catecholamin receptors, growth factor receptors, amino acid receptors or derivatives thereof, cytoline receptors, extracellular matrix molecule receptors, integrins, integrin receptors, hormones, hormone receptors, antibodies, lectines, cytokines, leptines, serpines, enzymes, proteases, kinases, phosphatases, hydrolases, trasncription factors, DNA binding proteins or peptides, RNA binding proteins or peptides, cell surface antigenes, virologe proteins such as HIV-protease or hepatitis C virus protease, or phages. The described procedure will be especially useful to design assays that present biological material on the tip of a wave guides.

1. Fabrication of surfaces with reduced protein adsorption and/or cell adhesion comprising a vapor deposition coating process such that the polymer includes chemical groups that reduce protein adsorption and/or cell adhesion.

2. Fabrication of surfaces with reduced protein adsorption and/or cell adhesion, wherein a functionalized polymer is provided that contains one or more different repetition units, where at least one of the repetition units is selected from the chemical structure (1) (as shown below): 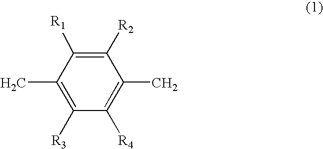
Rₙ (n=1,2,3,4) may be equal or different and may be selected from the group consisting of hydrogen, C1-C4 alkyl, aryl, amine, alcohol, ether, ethylene glycol, cyclic ether, thioether, crown ether, primary amide, secondary amide, ethylene glycol containing primary amide, ethylene glycol containing secondary amide, urethane, nitrile, isonitrile, nitrosamine, lactone, ethylene glycol containing urethane, carbamate, ethylene glycol containing carbamate, lactam, imine, hydrazone, ester, ethylene glycole containing ester, nitro compounds, nitrile, halo, organic radical, metalized group, acid halide group, isocyantate, thioisocyante, sulfur-containing groups (e.g. sulfonic acid, thioether, sulfonate, or sulfate ester group), silicon-containing group (e.g. silyl or silyloxy), or sugar derivatives.

3. Fabrication of surfaces with reduced protein adsorption and/or cell adhesion, wherein the coatings are based on poly[para-xylylenes]s or copolymers thereof.

4. Fabrication of surfaces with reduced protein adsorption and/or cell adhesion, wherein [2.2]paracyclophanes are polymerized during the chemical vapor deposition process, comprising activation of precursors at temperatures between 600 and 900° C. and pressures below 100 Pa and deposition of the polymers at temperatures below 160° C.

5. Method according to claim 2, wherein functional groups are provided that reduce protein adsorption and/or cell adhesion, said functional groups being incorporated in the functionalized polymer.

6. Method according to claim 2, wherein functional groups are provided, which allow chemical confinement of molecules that reduce protein adsorption and/or cell adhesion.

7. Method according to claim 6 comprising the use of spacer systems to confine molecules that reduce protein adsorption and/or cell adhesion to the surface.

8. Method according to claim 2, wherein a polymer is provided; said polymer being transparent.

9. Method according to claim 2, wherein a polymer is provided; said polymer having a thickness between 20 and 2000 nm.

10. Method according to claim 2, wherein functional groups are provided; said functional groups being used for confinement of molecules being able of capturing biomolecules.

11. Method according to claim 2, wherein functional groups are provided; said functional groups being used for confinement of molecules being able of capturing cells.

12. Method according to claim 10 comprising the use of spacer systems to confine capturing molecules to the surface.

13. Method according to claim 12 comprising the use of spacer systems to confine capturing molecules to the surface.

14. Method according to claim 12, wherein at least a part of the capturing molecules specifically binds to biomolecules that are subject to screening.

15. Method according to claim 14, wherein a surface is provided; said surfaces further comprising capturing molecules confined to the surface, which only temporarily bind at least a part of the biomolecules being subject to screening, thereby allowing their subsequent release.

16. Method according to claim 2, wherein functional groups are provided; said functional groups being used for confinement of temperature-sensitive molecules.

17. Method according to claim 2, wherein a polymer is provided; said polymer being further modified by plasma treatment.

18. Method according to claim 2, wherein a polymer is provided; said polymer being further modified by chemical treatment.

19. Method according to claim 2, wherein a polymer is provided; said polymer being further modified by treatment with a high energy source.

20. Method according to claim 4 comprising co-polymerization of [2.2]paracyclophanes with precursors of the general structures 84 and/or 85. 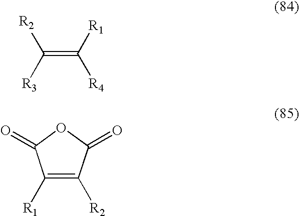
wherein Rₙ (n=1,2,3,4) may be equal or different and may be selected from the group consisting of hydrogen, C1-C4 alkyl, aryl, amine, alcohol, ether, ethylene glycol, cyclic ether, thioether, crown ether, primary amide, secondary amide, ethylene glycol containing primary amide, ethylene glycol containing secondary amide, urethane, nitrile, isonitrile, nitrosamine, lactone, ethylene glycol containing urethane, carbamate, ethylene glycol containing carbamate, lactam, imine, hydrazone, ester, ethylene glycole containing ester, nitro compounds, nitrite, halo, organic radical, metalized group, acid halide group, isocyantate, thioisocyante, groups of the general nature CO(O-M-A) (with M: C1-C4 aliphatic or aromatic group and A: e.g. hydrogen, hydroxyl-, amino-, or carboxy groups), sulfur-containing groups (e.g. sulfonic acid thioether, sulfonate, or sulfate ester group), silicon-containing group (e.g. silyl or silyloxy), or sugar derivatives.

21. Method according to claim 2 comprising a pre-treatment of the surface with plasma prior to deposition of the functionalized polymer.

22. A method for fabrication of bioassays comprising a) using a vapor deposition coating process to coat a substrate with a polymer film that reduces protein adsorption and/or cell adhesion, b) preparing a micro-patterned chemical array, c) modifying the micro-patterned chemical array by introducing an ordered array of capturing sides that bind to biologically relevant features, d) providing a fluidic delivery system for delivering at least one reagents to the ordered array, and e) providing a detection unit.

23. The method of claim 22 comprising a) using a vapor deposition coating process to coat a substrate with a polymer film that reduces protein adsorption and/or cell adhesion, b) preparing a micro-patterned chemical array, c) modifying the micro-patterned chemical array by introducing capturing sides that bind to cell features, d) binding at least one type of cells to the capturing sides to produce an ordered array of cell types, e) providing a fluidic delivery system for delivering a combinatorial of reagents to the ordered array of cell types, and f) providing a detection unit.

24. The method of claim 23, wherein said bioassay is conducted to assess cell migration of cells in response to exposure to at least one reagent.

25. The method of claim 23, wherein said bioassay is conducted to assess cell differentiation of cells exposed to a combinatorial to at least one reagent.

26. The method of claim 23, wherein said bioassay is conducted to assess cell invasion of cells exposed to a combinatorial to at least one reagent.

27. The method of claim 23, wherein said bioassay is conducted to assess cell motility of cells to a combinatorial to at least one reagent.

28. The method of claim 23, wherein said bioassay is conducted to assess cell apotosis of cells exposed to at least one reagent.

29. The method of claim 23, wherein said bioassay is conducted to assess cell proliferation of cells exposed to at least one reagent.

30. The method of claim 22 comprising a) using a vapor deposition coating process to coat a substrate with a polymer film that reduces protein adsorption and/or cell adhesion, b) preparing a micro-patterned chemical array, c) modifying the micro-patterned chemical array by introducing capturing sides that bind to biomolecules, d) binding biomolecules to the capturing sides to produce an ordered array of biomolecule types, e) providing a fluidic delivery system for delivering a combinatorial of at least one reagents to the ordered array of biomolecule types, and f) providing a detection unit.

31. The method of claim 30, wherein said bioassay is conducted to assess enzyme activity of biomoecules in response to exposure to at least one reagent.

32. The method of claim 30, wherein said bioassay is conducted to assess epitop mapping of biomolecules in response to exposure to at least one reagent.

33. The method of claim 30, wherein said bioassay is conducted to assess binding events between capturing biomolecules and biomolecules.

34. The method of claim 22 comprising coating of a substrate; said substrate being a microdevice made from polymers.

35. The method of claim 34 comprising coating of a substrate; said substrate being a microdevice made from an elastomeric polymer.

36. The method of claim 35 comprising coating of a substrate; said substrate being a microdevice made from polydimethylsiloxane.

37. The method of claim 34 comprising coating of a substrate; said substrate being a microdevice made from a poly(acrylate) and/or poly(methacrylate).

38. The method of claim 34 comprising coating of a substrate; said substrate being a microdevice made from glass, silicon and/or silicon dioxide.

39. The method of claim 2, wherein a functionalized polymer is deposited; said polymer covering only a part of the microdevice surface.

40. The method of claim 2, wherein more than one functionalized polymer is deposited at allocated regions of the surface.

41. The method of claim 2, wherein a functionalized polymer is deposited; said polymer providing an anisotropic distribution of chemical groups on the surface.

42. The method of claim 41, wherein a functionalized polymer is deposited; said polymer providing a chemical and/or biological gradient.
